(19) United States
(12) Patent Application Publication
(10) Pub. No.: US 2022/0175498 A1
(43) Pub. Date: Jun. 9, 2022

(54) DENTAL IMPLANT DETECTOR USING CONSTANT CURRENT SOURCE AND ITS DETECTING METHOD

(71) Applicant: Changsha Easyinsmile Intelligence Technology Co,Ltd, Changsha (CN)

(72) Inventors: DAN GUO, CHANGSHA (CN); TIRAN ZHANG, CHANGSHA (CN)

(21) Appl. No.: 17/241,811

(22) Filed: Apr. 27, 2021

(30) Foreign Application Priority Data
Dec. 4, 2020 (CN) 202011398342.5
Dec. 29, 2020 (CN) 202011597792.7

(51) Int. Cl. A61C 8/00; A61C 19/04

(52) U.S. Cl. CPC A61C 8/0093; A61C 19/04

(57) ABSTRACT

This invention specially refers to a dental implant detector using constant current source and its detecting method, which falls within the scope of anti-interference, high-sensitivity dental implant detection. It is composed of the detecting sensor, the detecting sensor socket and the implant locator. The detecting sensor socket is connected with the detecting sensor and the implant locator at two ends respectively. The detecting sensor is built with a PCB base and detecting coils mounted on the layer and plane of the PCB base. The involute racetrack copper coil on the plane of the PCB base is smaller than that on the layer by 0.1 mm both laterally and longitudinally. The implant locator is built mainly with a CPU with power supply, a constant voltage module, a status display control module and a frequency conversion module. The detector, highly sensitive, easy to operate and very accurate for location, has solved the problems like time-consuming, inaccuracy and slowness of traditional detection method. With it, the dentists are able to find the dental implant quickly and accurately before the operation, which helps reduce incidence of medical accidents.

### CROSS REFERENCE TO RELATED APPLICATION

This application claims the priority benefit of Chinese patent applications 202011398342.5, filed on Dec. 4, 2020, and 202011597792.7, filed Dec. 29, 2020, the entire disclosure of which is incorporated herein by reference in its entirety.

### TECHNICAL FIELD

This invention specially refers to a dental implant detector using constant current source and its detecting method, which falls within the scope of anti-interference, high-sensitivity dental implant detection.

### BACKGROUND TECHNOLOGIES

As people's living standards keep improving, more and more patients who are suffering from dental damages and losses choose to restore their chewing function with dental implants and crowns. After being used for a long time, the crown mounted on the dental implants in the oral cavity may become loose and fall out, then, muscle will grow at the wound and completely cover the implant. It is hard to use our bare eyes to find where on the jawbone the implants were placed. It is a very difficult technical problem to find the implants inserted in the jawbone and buried under the muscle. At the present, some doctors will use X-ray devices to take picture of the oral cavity for to find rough position of the implants. However, from time to time, the results are inaccurate because the X-ray device can only take normal sectional picture of the teeth. If there are dental implants and anchorage nails very close to each other in the oral cavity, or there are multiple implants among the external teeth and molar teeth, images of the implants will be overlapped or distorted in the X-ray picture, and it will be very hard to find accurate positions of the implants and may bring about medical accidents. Therefore, how to find all dental implants and their accurate positions in the oral cavity before an operation is a technical problem which demands an urgent solution.

When people's teeth fall out, generally, we need to fix the dental implants on the gum and mount tooth crowns on the implants for the artificial teeth to exert chewing function. The crowns mounted on the implants generally have a service life of about 5 years, after which, they would fall out. Then, the muscle on the gum will grow and cover the implant. When people go to a dentist after some time, it is probable that the dentist cannot find the implant in the gum. Therefore, it is very important to identify the dental implants in the upper or lower jaw and find their positions accurately and quickly. This invention is a PCB-based anti-interference dental implant probe, which can not only identify dental implants in the upper or lower jaw but also find their accurate positions.

### INVENTION DESCRIPTION

The purpose of this invention is to build a highly sensitive dental implant probe, which specifically refers to a dental implant detector using constant current source and its detecting method, so as to guarantee portability of the detector and its accuracy of detection.

This invention is realized by the following technical blueprint. It is composed of the detecting sensor, the detecting sensor socket and the implant locator. The detecting sensor socket is connected with the detecting sensor and the implant locator at two ends respectively.

The detecting sensor is built with a PCB base, detecting coils mounted on the layer and plane of the PCB base, the center hole at the middle of the base and the via holes which connect the coils on the layer and plane of the base. The detecting coils are two involute racetrack copper coils mounted on the layer and plane of the PCB base. The coil on the plane is smaller than that on the layer by 0.1 mm both laterally and longitudinally. Electricity is supplied to the coils through the electrode connected with the power supply.

The implant locator is built mainly with a CPU with power supply, a constant voltage module, a status display control module and a frequency conversion module.

The detecting sensor socket is connected with the detecting sensor at one end and with the front tip of the implant locator at the other end. The PCB is connected to power supply through the detecting sensor socket.

The constant voltage module, status display control module and the frequency conversion module are connected with the CPU. The PCB is connected with the CPU through the detecting sensor socket.

The frequency conversion module controlled by the CPU will produce different frequency ranges according to the gears chosen and implement scanning with increasing frequency within the chosen range.

The PCB base is made with glass fiber cloth material (FR-4), which can be 1.0 mm, 1.2 mm or 1.5 mm thick.

The copper line constituting a coil is 35 um thick and 0.2 mm-0.25 mm wide. The interval between two neighboring coil rings is 0.1 mm-0.15 mm. The detecting coil on the layer of the PCB base has 6 rings, and the inner ring is smaller than the external ones. This coil is connected through a via hole with the coil on the plane of the PCB base. The coil on the plane is also made with 6 rings which become larger from inside out. The center line of the coil on the plane corresponds in position with the center line of intervals between coil rings on the layer.

The detecting coil on the layer measures 5.5 mm-6.6 mm longitudinally and 6.5 mm-7.6 mm laterally. The straight part of a ring of the involute racetrack coil is 2 mm-3 mm long.

From inside out, the diameter of the semicircular part of the involute racetrack coil is 2.5 mm, 3.1 mm, 3.7 mm, 4.3 mm, 4.9 mm and 5.5 mm, with a positive variation of 0-0.1 mm.

From inside out, lateral dimension of the involute racetrack coil is 3.5 mm, 4.1 mm, 4.7 mm, 5.3 mm, 5.9 mm and 6.5 mm, with a positive variation of 0-0.1 mm. The center hole on the PCB Base measures 2 mm-3 mm in diameter, which is laminated with copper around.

The three via holes connecting coils on the layer and plane of the PCB base measure 500 um-700 um in diameter, and are distributed along an obtuse triangle.

The detecting coil on the layer of PCB base produces concave magnetic field distribution. The sinusoidal AC flowing through the above coil can be adjusted between 230 kHz and 240 kHz in frequency, and its peak voltage will fluctuate between 400 mV and 450 mV during normal work.

The detecting sensor socket can rotate 0°-90°, 90°-180° and 180°-270° inside the implant locator. The detecting sensor socket is inserted into the plughole of the implant locator.

The plugging end of the detecting sensor socket takes a cylindrical shape, which has a limit boss at 0° position. There are rhombic spring leaves covering the plugging end on the outside. The spring leaves are locked inside the plughole by limit steps set up on the inner wall of the plughole, where, locating slots are also used to harbor 4 corners of the rhombic spring leaves. The angle between two neighboring limit steps is 90°.

The detecting sensor has a hollow structure and there are electrical cables mounted inside.

The status display control module is located where the implant locator bends. The module is split into 8 parts, with each part being equipped with a red LED light and a green one. The 8 parts are display areas 0-5 and key 1 and key 2. The circular display area 0 is positioned at the center, around which surround Key 1 and key 2. Then, there is a circular area comprising display areas 1-5 outside Key 1 and Key 2.

It is powered by battery, which is mounted inside the battery compartment at the end of implant locator and provides electricity to both the detecting sensor and the implant locator.

When the battery voltage nears its critical point, the constant voltage module controlled by the CPU will turn on the constant voltage circuit to provide for normal function of the device and reduce under-performance time. When battery voltage falls below the threshold value, the device cannot start and a reminder of replacing the battery shows up.

The frequency conversion module has 3 gears: the high gear of a 8-12 MHz frequency range, the intermediate gear of 4-8 MHz, and the low gear of 0.1-4 MHz. A 300 um thick copper mesh shield is embedded on the inner wall of the implant locator.

A dental implant detecting method using constant current source:

Step 1. put a battery inside the battery compartment of the implant locator, insert the detecting sensor and the sensor socket into the front end of the implant locator.

Step 2. press Key 2, the buzzer gives a short beep, green LED lights at display areas 0-5 will be lighted for 0.5 second before going off, after which, the implant locator starts self-checking. If the locator fails in self-checking, display areas 1-5 will light the red LED lights successively with an interval of 0.2 second before the locator turns off automatically in 1 second. If the locator passes self-checking, display areas 1-5 will light the green LED lights constantly.

Step 3. press Key 1, the system enters into treatment mode, display areas 0-5 twinkle their green LED lights simultaneously with an interval of 0.5 second in a cyclic way. When the detecting sensor enters a moderate or weak detecting area, green and red LED lights at display areas 0-5 will be turned on simultaneously to show an orange color. When the detecting sensor enters a strong detecting area, display area 0 will ignite its green LED light and the buzzer will give off two long beeps, which indicates the implant is found. After marking the position, one can continue to find the next implant.

During the above step 3, the following frequency conversion operations are also implemented:

Send the detecting sensor into the oral cavity, rotate the detecting sensor socket to get the best angle for carrying out detection. Press down the treatment key, namely Key 1. Shift to the low frequency gear and move the sensor over tooth surface. If the sensor enters into the implantation area, display areas 1-5 will be lighted up with an orange color due to the green and red lights being turned on simultaneously. Continue to move the sensor, and when the sensor comes to the center of the implantation area, the green LED light goes on at display area 0 while lights at other display areas are off. If no implant is found, shift to intermediate or high gears for higher frequency produced by the frequency conversion module under control of CPU to re-detect until the implant is found.

The above-mentioned step 3 also include the following detecting sub-steps:

Step 3.1. insert the coil-mounting PCB base into probe of the detector and send the probe into vicinity of teeth in oral cavity.

Step 3.2. start power supply to the detector, high-frequency electrical current will flow through the detecting coil and produce around the coil a magnetic field with periodical oscillation.

Step 3.3. when the magnetic field interacts with the metallic dental implant buried under the gum, it will produce induced electromotive force inside the implant, as a result of which, an induced current is generated. However, magnetic field of the induced current will interfere with the original magnetic field around the coil to bring about changes to electrical current in the coil. The testing device from the detector will give signals to warn of changes of the current.

Since coils on the layer and plane of the PCB base are asymmetric, their magnetic fields differ in intensity. Even at the middle between teeth of the upper and lower jaws, the probe can still detect the metallic implant quickly, and then locate it accurately by moving the probe.

Magnetic induction intensity of the involute racetrack coils in above-mentioned step 3 is calculated with the formula:

$$\begin{matrix}\vec{B} ( z ) = \frac{{μ}_{0} I}{4 π} {∳}_{l} \frac{{dl}^{′} \times \vec{R}}{{R}^{3}} \\ = \frac{{μ}_{0} Ia}{4 π {R}^{2}} [ {\int }_{0}^{2 π} {\vec{e}}_{φ} \times ( - {e}_{r} sin α + {e}_{z} cos α ) d {φ}^{′} ] \\ = \frac{{μ}_{0} Ia}{4 π {R}^{2}} ( {\vec{e}}_{z} sin α {\int }_{0}^{2 π} d {φ}^{′} + cos α {\int }_{0}^{2 π} {\vec{e}}_{r} d {φ}^{′} ) \\ = {\vec{e}}_{z} \frac{{μ}_{0} {Ia}^{2}}{2 {R}^{3}} \\ = {\vec{e}}_{z} \frac{{μ}_{0} {Ia}^{2}}{{2 [ {a}^{2} + {z}^{2} ]}^{3 / 2}}\end{matrix}$$

Taking coil on the layer as an xoy plane, origin of coordinates is at the middle of the center hole, therefore, magnetic field distribution along the central axis above the xoy plane is calculated with the formula:

$${\vec{B} ( z )}_{\text{?}} = {\vec{e}}_{z} \frac{{μ}_{0} {Ia}^{2}}{2} [ \frac{1}{{[ {a}^{2} + {z}^{2} ]}^{\frac{3}{2}}} + \frac{1}{{[ {( a + Δ a )}^{2} + {z}^{2} ]}^{\frac{3}{2}}} + \frac{1}{{[ {( a + 2 Δ a )}^{2} + {z}^{2} ]}^{\frac{3}{2}}} + \frac{1}{{[ {( a + 3 Δ a )}^{2} + {z}^{2} ]}^{\frac{3}{2}}} + \frac{1}{{[ {( a + 4 Δ a )}^{2} + {z}^{2} ]}^{\frac{3}{2}}} + \frac{1}{{[ {( a + 5 Δ a )}^{2} + {z}^{2} ]}^{\frac{3}{2}}} ]$$

$$\text{?} \text{indicates text missing or illegible when filed}$$

Magnetic field distribution along the central axis below the xoy plane is calculated with the formula:

$${\vec{B} ( z )}_{\text{?}} = {\vec{e}}_{z} \frac{{μ}_{0} {Ia}^{2}}{2} [ \frac{1}{{[ {( a + Δ a )}^{2} + {( z + d )}^{2} ]}^{\frac{3}{2}}} + \frac{1}{{[ {( a + 2 Δ a )}^{2} + {( z + d )}^{2} ]}^{\frac{3}{2}}} + \frac{1}{{[ {( a + 3 Δ a )}^{2} + {( z + d )}^{2} ]}^{\frac{3}{2}}} + \frac{1}{{[ {( a + 4 Δ a )}^{2} + {( z + d )}^{2} ]}^{\frac{3}{2}}} + \frac{1}{{[ {( a + 5 Δ a )}^{2} + {( z + d )}^{2} ]}^{\frac{3}{2}}} + \frac{1}{{[ {( a + 6 Δ a )}^{2} + {( z + d )}^{2} ]}^{\frac{3}{2}}} ]$$

$$\text{?} \text{indicates text missing or illegible when filed}$$

Finally, overall magnetic field distribution along the central axis is calculated with the formula:

$${\vec{B} ( z )}_{\text{?}} = {\vec{e}}_{z} \frac{{μ}_{0} {Ia}^{2}}{2} [ \frac{1}{{[ {a}^{2} + {z}^{2} ]}^{\frac{3}{2}}} + \frac{1}{{[ {( a + Δ a )}^{2} + {z}^{2} ]}^{\frac{3}{2}}} + \frac{1}{{[ {( a + 2 Δ a )}^{2} + {z}^{2} ]}^{\frac{3}{2}}} + \\ \frac{1}{{[ {( a + 3 Δ a )}^{2} + {z}^{2} ]}^{\frac{3}{2}}} + \frac{1}{{[ {( a + 4 Δ a )}^{2} + {z}^{2} ]}^{\frac{3}{2}}} + \frac{1}{{[ {( a + 5 Δ a )}^{2} + {z}^{2} ]}^{\frac{3}{2}}} ] + \\ + {\vec{e}}_{z} \frac{{μ}_{0} {Ia}^{2}}{2} [ \frac{1}{{[ {( a + Δ a )}^{2} + {( z + d )}^{2} ]}^{\frac{3}{2}}} + \frac{1}{{[ {( a + 2 Δ a )}^{2} + {( z + d )}^{2} ]}^{\frac{3}{2}}} + \frac{1}{{[ {( a + 3 Δ a )}^{2} + {( z + d )}^{2} ]}^{\frac{3}{2}}} + \frac{1}{{[ {( a + 4 Δ a )}^{2} + {( z + d )}^{2} ]}^{\frac{3}{2}}} + \frac{1}{{[ {( a + 5 Δ a )}^{2} + {( z + d )}^{2} ]}^{\frac{3}{2}}} + \frac{1}{{[ {( a + 6 Δ a )}^{2} + {( z + d )}^{2} ]}^{\frac{3}{2}}} ]$$

$$\text{?} \text{indicates text missing or illegible when filed}$$

Benefits of this invention include: 1. the dental-implant-detecting coils of this invention are optimized by computer simulation and theoretical calculation, which can be built easily with low cost; 2. since it is used inside the oral cavity, the low cost makes it disposable, so as to prevent the coils from being contaminated by bacteria in the oral cavity and block the transmission of bacteria; 3. electrical current in the coils is only about 20 mA, and the alternating magnetic field produced will not injure the biological tissue in the oral cavity; 4. the detecting structure of one involute racetrack copper coil on two sides of the PCB base guarantees both portability and accuracy of detection; 5. different gears entail varying energy consumption, which can help save more energy; 6. there is a frequency conversion module in the locator, which under control of the single-chip microprocessor can output certain frequency range corresponding to the gear chosen and carry out scanning operation with increasing frequency within the chosen range; 7. the copper mesh shield can reduce interference from external signals. Circuits in the device also adopt relevant measures, like using qualifying low-speed chip and optical couplers to prevent signal crosstalk. Anti-interference measures are also used on power supply, PCB routing, grounding, cable connecting. Therefore, the device has very good anti-interference capability; 8. the detector has different models, from which the most suitable will be used to cater to conditions of the patients; 9. Combination of the on/off status of the diodes emitting red or green color corresponds with the status of the detector, which can help locate quickly; 10. the sensor socket on the locator can be adjusted manually to rotate the sensor around the locator to facilitate operation; 11. output of the locator is low-frequency AC signal, which will not result in radiation injury to any part of the oral cavity.

### ILLUSTRATION PICTURES

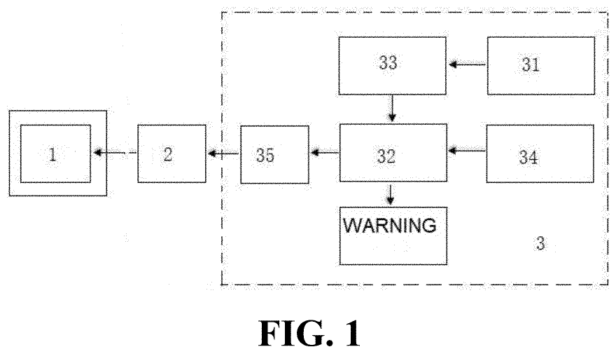

FIG. 1. Structure of the Implant Locator

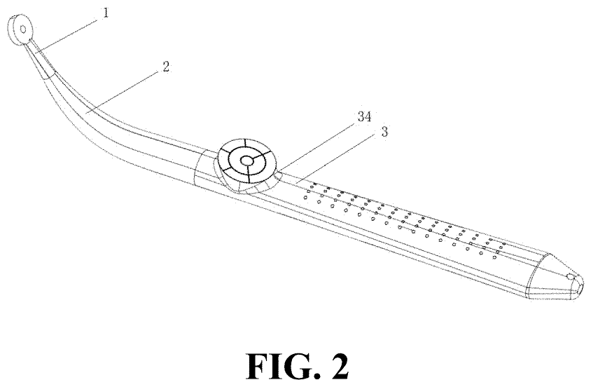

FIG. 2. Structure Diagram of the Invention

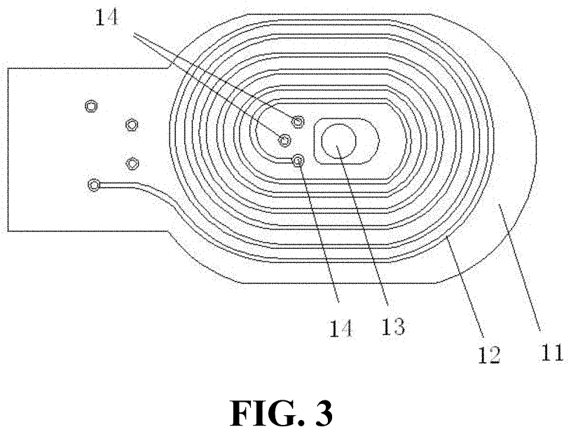

FIG. 3. Front View of the Probe

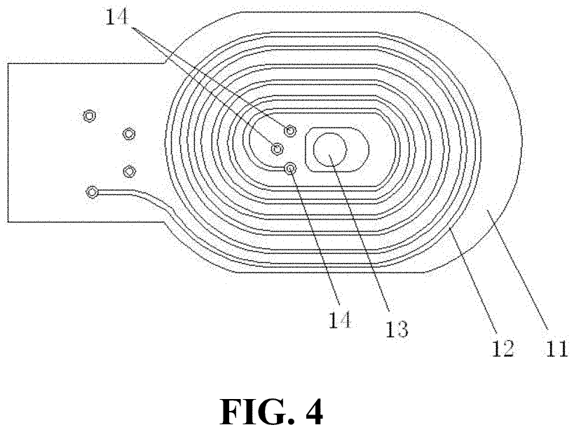

FIG. 4. Back View of the Probe

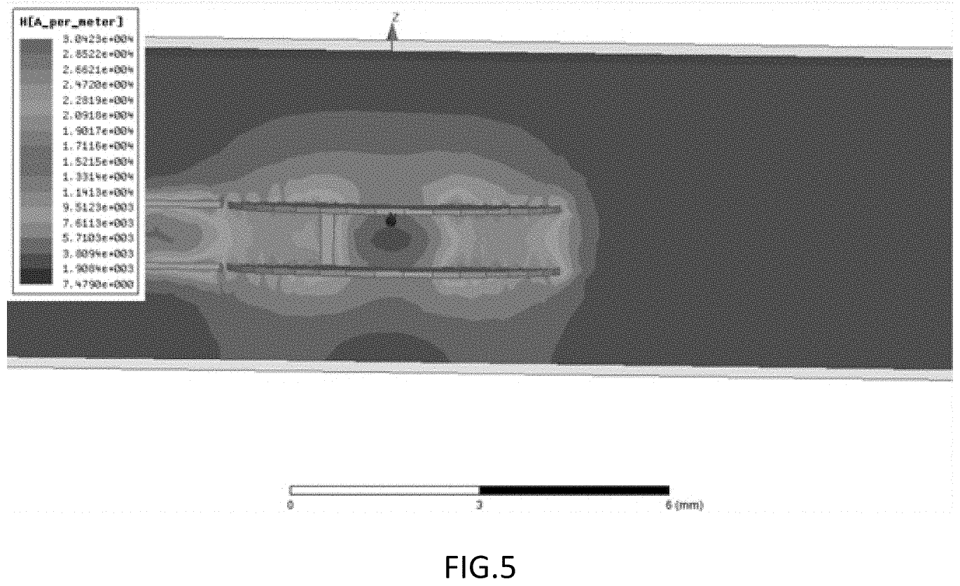

FIG. 5. Simulation of Magnetic Field Distribution around the Probe

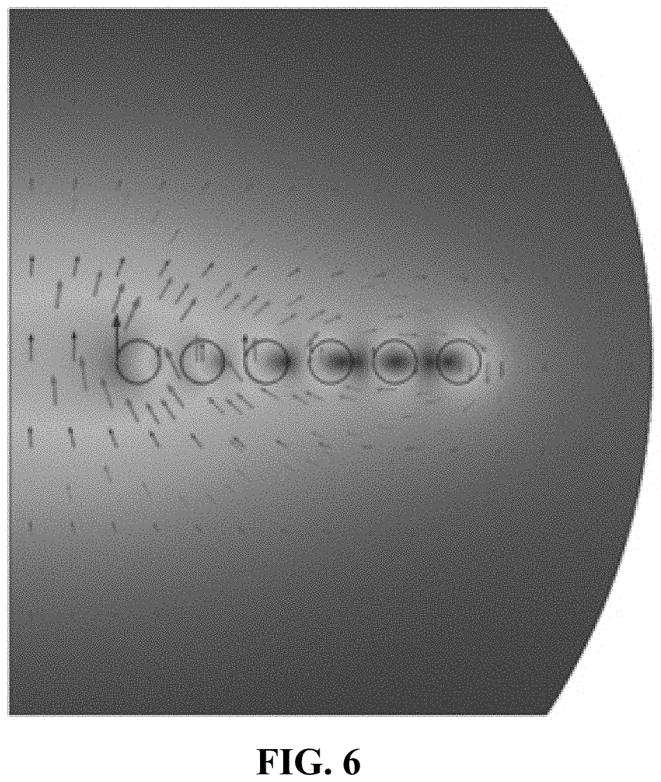

FIG. 6. Magnetic Field Distribution of Coils in the Front Probe

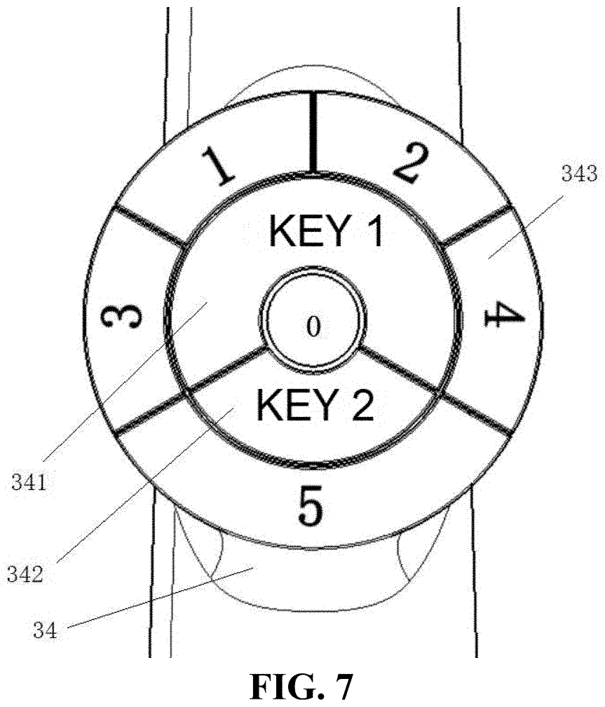

FIG. 7. Status Display Control Module Sketch

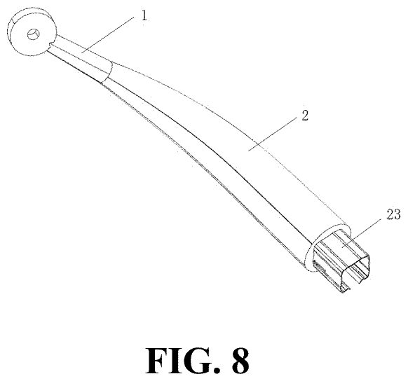

FIG. 8. Detecting Sensor Socket Sketch

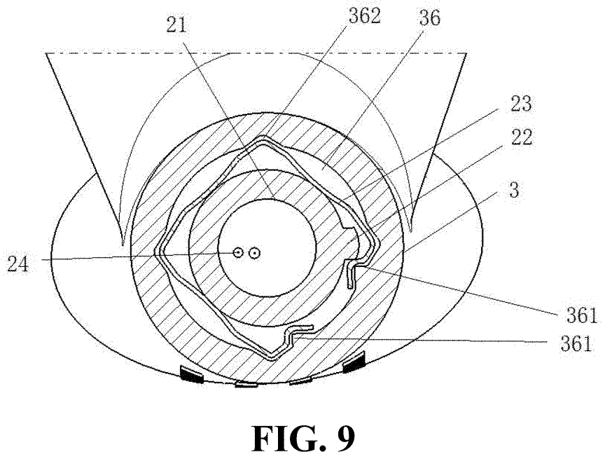

FIG. 9. Section View of the Detecting Sensor Socket

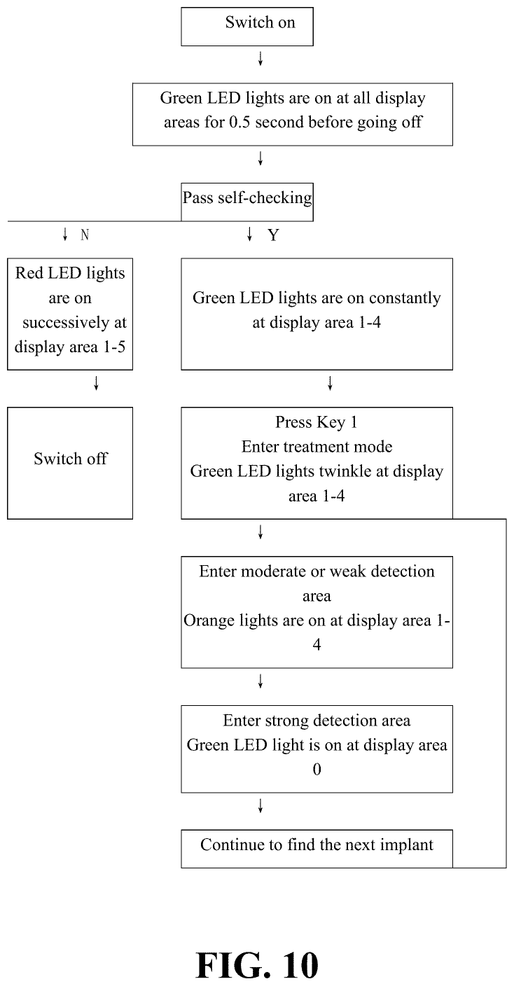

FIG. 10. Flowchart of the Invention

### WAY OF IMPLEMENTATION

Next, by means of FIG. 1-10 appended herein, an elaborate and complete description is given of the technological blueprint of an embodiment of this invention. Obviously, the embodiment described herein is only a part of examples, not all of the this invention. Any other embodiment developed on the basis of this embodiment of the invention and without additional creative work by ordinary technicians in the same field shall be protected under the umbrella of this invention.

This invention is composed of the detecting sensor(1), the detecting sensor socket (2) and the implant locator(3). The detecting sensor socket is connected with the detecting sensor(1) and the implant locator (3) at two ends respectively.

The detecting sensor (1) is built with a PCB base (11), detecting coils (12) mounted on the layer and plane of the PCB base (11), the center hole (13) at the middle of the base (11) and the via holes (14) which connect the coils (12) on the layer and plane of the base (11). The detecting coils (12) are two involute racetrack copper coils mounted on the layer and plane of the PCB base (11). The coil on the plane is smaller than that on the layer by 0.1 mm both laterally and longitudinally. Electricity is supplied to the detecting coils (12) through the electrode connected with the power supply. There are different types of detecting coils (1), when the PCB keep constant in size, we can change the number of rings of the coils mounted on the PCB to meet requirements of different people.

The implant locator (3) is built mainly with a CPU (32) with power supply (31), a constant voltage module (33), a status display control module (34) and a frequency conversion module (35). The implant locator (3), the core of this device, has a hand-held structure, which is cylindrical, 12 cm long and 2 cm across. The detecting sensor socket (2) bends 30° at 2 cm from the top, the diameter of which dwindles gradually and becomes 1.8 cm at the end port, where a plug that can be rotated 0°-270° is mounted to connect with the detecting sensor.

There are rubber humps embedded on the front, back and sides of the outer shell of the implant locator (3), which can increase friction and prevent the locator from falling out.

The detecting sensor socket (2) is connected with the detecting sensor (1) at one end and with the front part of the implant locator (3) at the other end. The PCB is connected with power supply (31) through the detecting sensor socket (2).

The constant voltage module (33), status display control module (34) and the frequency conversion module (35) are connected with the CPU (32). The PCB is connected with the CPU (32) through the detecting sensor socket (2).

When in use, the detecting sensor (1) after detecting the implant in the oral cavity will instantly translate electromagnetic signals into level signals and transmit them to CPU (32). CPU (32) will receive, process and analyze the signals, and identify the implant after removing interfering signals, after which, the status display module (34) driven by power supply (31) will register the status accordingly.

The frequency conversion module (35) controlled by the CPU (32) will produce different frequency ranges according to the gears chosen and implement scanning with increasing frequency within the chosen range.

The detecting theory is: Correlation among the electrical inductance L₁, electric resistance R₁, voltage effective value E, current effective value I₁, angular frequency ω, of the detecting coils, the equivalent eddy inductance L₂, electric resistance R₂, eddy effective value I₂ of the metallic implant, and the mutual inductance M between the detecting coil and metallic implant can be expressed in formulas:

I₁R₁+jL₁ωI₁=E+jMωI₂ (1)

jMωI₁=I₂R₂+jLωI₂ (2)

From formula (1) and (2) we get another formula:

$$E = {I}_{1} [ ( {R}_{1} + \frac{{M}^{2} {ω}^{2} {R}_{2}}{{R}_{2}^{2} + {ω}^{2} {L}_{2}^{2}} ) + j ω ( {L}_{1} - \frac{{M}^{2} ω {L}_{2}}{{R}_{2}^{2} + {ω}^{2} {L}_{2}^{2}} ) ]$$

When the detecting sensor (1) gets close to a metallic implant in the oral cavity, the eddy effect will change the equivalent electrical inductance and equivalent electrical resistance of the detecting coils, which in turn will bring about voltage change to the detecting coils and signify that the implant is found.

The PCB base (11) is made with glass fiber cloth material (FR-4), which can be 1.0 mm, 1.2 mm or 1.5 mm thick.

The copper line constituting a coil (12) is 35 um thick and 0.2 mm-0.25 mm wide. The interval between two neighboring coil rings is 0.1 mm-0.15 mm. The detecting coil (12) on the layer of the PCB base (11) has 6 rings, which become smaller from outside in. This coil (12) is connected through a via hole (14) with the coil (12) on the plane of the PCB base (11). The coil (12) on the plane is also made with 6 rings which become larger from inside out. The center line of the coil (12) on the plane corresponds in position with the center line of intervals between rings of the coil (12) on the layer.

The detecting coil (12) on the layer measures 5.5 mm-6.6 mm longitudinally and 6.5 mm-7.6 mm laterally. The straight part of a ring of the involute racetrack coil is 2 mm-3 mm long.

From inside out, the diameter of the semicircular part of the involute racetrack coil is 2.5 mm, 3.1 mm, 3.7 mm, 4.3 mm, 4.9 mm and 5.5 mm, with a positive variation of 0-0.1 mm.

From inside out, lateral dimension of the involute racetrack coil is 3.5 mm, 4.1 mm, 4.7 mm, 5.3 mm, 5.9 mm and 6.5 mm, with a positive variation of 0-0.1 mm.

The center hole (13) on the PCB Base (11) measures 2 mm-3 mm in diameter, which is laminated with copper around.

The three via holes (14) connecting coils (12) on the layer and plane of the PCB base (11) measure 500 um-700 um in diameter, and are distributed along an obtuse triangle.

The detecting coil (12) on the layer of PCB base (11) produces concave magnetic field distribution. The sinusoidal AC flowing through the above coils (12) can be adjusted between 230 kHz and 240 kHz in frequency, and its peak voltage will fluctuate between 400 mV and 450 mV during normal work.

The CPU (32) is a single-chip microprocessor, the working voltage of which is 1.3V-1.5V. It is a ULP microprocessor, which is powered directly by the battery.

The detecting sensor socket (2) can rotate 0°-90°, 90°-180° and 180°-270° inside the implant locator (3). The detecting sensor socket (2) is inserted into the plughole (36) of the implant locator (3) through plugging end(21).

The plugging end (21) of the detecting sensor socket (2) takes a cylindrical shape, which has a limit boss (22) at 0° position. There are rhombic spring leaves (23) covering the plugging end (21) on the outside. The spring leaves (23) are locked inside the plughole (36) by limit steps (361) set up on the inner wall of the plughole (36), where, locating slots (362) are also used to harbor 4 corners of the rhombic spring leaves (23). The angle between two neighboring limit steps (361) is 90°.

The detecting sensor socket (2) has a hollow structure and there are electrical cables (24) mounted inside.

The detecting sensor socket (2) can rotate 0°-270°, to facilitate detection of the implants in the upper and lower jaws. The detecting sensor socket (2) has electrodes at both ends, which guarantees that the device functions no mater the socket (2) is inserted in normally or reversely, so as to save time in replacing the socket (2).

The status display control module (34) is located where the implant locator bends. The module (34) is split into 8 parts, with each part being equipped with a red LED light and a green one. The 8 parts are display areas 0-5 (343) and key 1 (341) and key 2 (342). The circular display area 0 is positioned at the center, around which surround Key 1 (341) and key 2 (342). Then, there is a circular area comprising display areas 1-5 outside Key 1 and Key 2.

It is powered by battery (31), which is mounted inside the battery compartment at the end of implant locator (3) and provides electricity to both the detecting sensor (1) and the implant locator (3).

When the battery voltage nears its critical point, the constant voltage module (33) controlled by the CPU (32) will turn on the constant voltage circuit to provide for normal function of the device and reduce under-performance time. When battery voltage falls below the threshold value, the device cannot start and a reminder of replacing the battery shows up. This device is more convenient and quicker than existent X-ray machine on locating the implant and it will not bring radiation injury to people.

The frequency conversion module (35) has 3 gears: the high gear of a 8-12 MHz frequency range, the intermediate gear of 4-8 MHz, and the low gear of 0.1-4 MHz.

A 300 um thick copper mesh shield is embedded on the inner wall of the implant locator (3) to reduce interference from external signals. Circuits in the implant locator (3) also adopt relevant measures, like using qualifying low-speed chip and optical couplers to prevent signal crosstalk. Anti-interference measures are also used on power supply, PCB routing, grounding, cable connecting. Therefore, the device has very good anti-interference capability.

The following steps must be followed to operate the invention:

Step 1. put a AA battery inside the battery compartment of the implant locator (3), insert the detecting sensor (1) and the sensor socket (2) into the front end of the implant locator(3).

Step 2. press Key 2 (342), the buzzer gives a short beep, green LED lights at display areas 0-5 (343) will be lighted for 0.5 second before going off, after which, the implant locator (3) starts self-checking. If the locator fails in self-checking, display areas 1-5 (343) will light the red LED lights successively with an interval of 0.2 second before the locator turns off automatically in 1 second. If the locator passes self-checking, display areas 0-5 (343) will light the green LED lights constantly.

Step 3. press Key 1 (341), the system enters into treatment mode, display areas 0-5 (343) twinkle their green LED lights simultaneously with an interval of 0.5 second in a cyclic way. When the detecting sensor enters a moderate or weak detecting area, green and red LED lights at display areas 0-5 (343) will be turned on simultaneously to show an orange color. When the detecting sensor (1) enters a strong detecting area, display area 0 will ignite its green LED light and the buzzer will give off two long beeps, which indicates the implant is found. After marking the position, one can continue to find the next implant.

During the above step 3, the following frequency conversion operations are also implemented:

Send the detecting sensor (1) into the oral cavity, rotate the detecting sensor socket (2) to get the best angle for carrying out detection. Press down the treatment key, namely Key 1 (341). Shift to the low frequency gear and move the sensor over tooth surface. If the sensor enters into the implantation area, display areas 1-5 (343) will be lighted up with an orange color due to the green and red lights turned on simultaneously. Continue to move the sensor, and when the sensor comes to the center of the implantation area, the green LED light goes on at display area 0 while lights at other display areas are off. If no implant is found, shift to intermediate or high gears for higher frequency produced by the frequency conversion module (35) under control of CPU (32) to re-detect until the implant is found.

The above-mentioned step 3 also include the following detecting sub-steps:

Step 3.1. insert the coil-mounting PCB base (11) into probe of the detector and send the probe into vicinity of teeth in oral cavity.

Step 3.2. start power supply to the detector, high-frequency electrical current will flow through the detecting coil (12) and produce around the coil a magnetic field with periodical oscillation.

Step 3.3. when the magnetic field interacts with the metallic dental implant buried under the gum, it will produce induced electromotive force inside the implant, as a result of which, an induced current is generated. However, magnetic field of the induced current will interfere with the original magnetic field around the coil to bring about changes to electrical current in the coil (12). The testing device from the detector will give signals to warn of changes of the current.

Since coils (12) on the layer and plane of the PCB base (11) are asymmetric, their magnetic fields differ in intensity. Even at the middle between teeth of the upper and lower jaws, the probe can still detect the metallic implant quickly, and then locate it accurately by moving the probe.

Magnetic induction intensity of the involute racetrack coils in above-mentioned step 3 is calculated with the formula:

$$\begin{matrix}\vec{B} ( z ) = \frac{{μ}_{0} I}{4 π} {∳}_{l} \frac{{dl}^{′} \times \vec{R}}{{R}^{3}} \\ = \frac{{μ}_{0} Ia}{4 π {R}^{2}} [ {\int }_{0}^{2 π} {\vec{e}}_{φ} \times ( - {e}_{r} sin α + {e}_{z} cos α ) d {φ}^{′} ] \\ = \frac{{μ}_{0} Ia}{4 π {R}^{2}} ( {\vec{e}}_{z} sin α {\int }_{0}^{2 π} d {φ}^{′} + cos α {\int }_{0}^{2 π} {\vec{e}}_{r} d {φ}^{′} ) \\ = {\vec{e}}_{z} \frac{{μ}_{0} {Ia}^{2}}{2 {R}^{3}} \\ = {\vec{e}}_{z} \frac{{μ}_{0} {Ia}^{2}}{{2 [ {a}^{2} + {z}^{2} ]}^{3 / 2}}\end{matrix}$$

Taking coil on the layer as an xoy plane, origin of coordinates is at the middle of the center hole (13), therefore, magnetic field distribution along the central axis above the xoy plane is calculated with the formula:

$${\vec{B} ( z )}_{up} = {\vec{e}}_{z} \frac{{μ}_{0} {Ia}^{2}}{2} [ \frac{1}{{[ {a}^{2} + {z}^{2} ]}^{\frac{3}{2}}} + \frac{1}{{[ {( a + Δ a )}^{2} + {z}^{2} ]}^{\frac{3}{2}}} + \frac{1}{{[ {( a + 2 Δ a )}^{2} + {z}^{2} ]}^{\frac{3}{2}}} + \frac{1}{{[ {( a + 3 Δ a )}^{2} + {z}^{2} ]}^{\frac{3}{2}}} + \frac{1}{{[ {( a + 4 Δ a )}^{2} + {z}^{2} ]}^{\frac{3}{2}}} + \frac{1}{{[ {( a + 5 Δ a )}^{2} + {z}^{2} ]}^{\frac{3}{2}}} ]$$

Magnetic field distribution along the central axis below the xoy plane is calculated with the formula:

$${\vec{B} ( z )}_{down} = {\vec{e}}_{z} \frac{{μ}_{0} {Ia}^{2}}{2} [ \frac{1}{{[ {( a + Δ a )}^{2} + {( z + d )}^{2} ]}^{\frac{3}{2}}} + \frac{1}{{[ {( a + 2 Δ a )}^{2} + {( z + d )}^{2} ]}^{\frac{3}{2}}} + \frac{1}{{[ {( a + 3 Δ a )}^{2} + {( z + d )}^{2} ]}^{\frac{3}{2}}} + \frac{1}{{[ {( a + 4 Δ a )}^{2} + {( z + d )}^{2} ]}^{\frac{3}{2}}} + \frac{1}{{[ {( a + 5 Δ a )}^{2} + {( z + d )}^{2} ]}^{\frac{3}{2}}} + \frac{1}{{[ {( a + 6 Δ a )}^{2} + {( z + d )}^{2} ]}^{\frac{3}{2}}} ]$$

Finally, overall magnetic field distribution along the central axis is calculated with the formula:

$${\vec{B} ( z )}_{total} = {\vec{e}}_{z} \frac{{μ}_{0} {Ia}^{2}}{2} [ \frac{1}{{[ {a}^{2} + {z}^{2} ]}^{\frac{3}{2}}} + \frac{1}{{[ {( a + Δ a )}^{2} + {z}^{2} ]}^{\frac{3}{2}}} + \frac{1}{{[ {( a + 2 Δ a )}^{2} + {z}^{2} ]}^{\frac{3}{2}}} + \frac{1}{{[ {( a + 3 Δ a )}^{2} + {z}^{2} ]}^{\frac{3}{2}}} + \frac{1}{{[ {( a + 4 Δ a )}^{2} + {z}^{2} ]}^{\frac{3}{2}}} + \frac{1}{{[ {( a + 5 Δ a )}^{2} + {z}^{2} ]}^{\frac{3}{2}}} ] + {\vec{e}}_{z} \frac{{μ}_{0} {Ia}^{2}}{2} [ \frac{1}{{[ {( a + Δ a )}^{2} + {( z + d )}^{2} ]}^{\frac{3}{2}}} + \frac{1}{{[ {( a + 2 Δ a )}^{2} + {( z + d )}^{2} ]}^{\frac{3}{2}}} + \frac{1}{{[ {( a + 3 Δ a )}^{2} + {( z + d )}^{2} ]}^{\frac{3}{2}}} + \frac{1}{{[ {( a + 4 Δ a )}^{2} + {( z + d )}^{2} ]}^{\frac{3}{2}}} + \frac{1}{{[ {( a + 5 Δ a )}^{2} + {( z + d )}^{2} ]}^{\frac{3}{2}}} + \frac{1}{{[ {( a + 6 Δ a )}^{2} + {( z + d )}^{2} ]}^{\frac{3}{2}}} ]$$

It must be clearly stated that terms like “include”, “comprise” and their equivalents shall mean non-exclusive inclusion, therefore, a process, method, article or equipment made with a certain list factors will also include other unlisted factors or factors inherent in the process, method, article or equipment. If with no more restrictions, any factor restricted by the word “including one” will not exclude its equivalents which might exist in the process, method, article or equipment involving the said factor. Although the embodiment of the present invention has been shown and described, for ordinary skilled personnel in the field, it can be understood that the embodiment may be changed, modified, replaced and varied without leaving the principle and spirit of the present invention, and the scope of the present invention is limited by the attached claims and their equivalents.

We claim:

1. A dental implant detector using constant current source comprising:
a detecting sensor, a detecting sensor socket and an implant locator; the detecting sensor socket is connected with the detecting sensor and the implant locator at two ends respectively;
the detecting sensor is built with a PCB base, detecting coils mounted on the layer and plane of the PCB base, a center hole at the middle of the base and via holes which connect the coils on the layer and plane of the base; the detecting coils are two involute racetrack copper coils mounted on the layer and plane of the PCB base; the coil on the plane is smaller than that on the layer by 0.1 mm both laterally and longitudinally; electricity is supplied to the coils through the electrode connected with the power supply; the implant locator is built mainly with a CPU with power supply, a constant voltage module, a status display control module and a frequency conversion module;
the detecting sensor socket is connected with the detecting sensor at one end and with the front tip of the implant locator at the other end; the PCB is connected to power supply through the detecting sensor socke;
the constant voltage module, status display control module and the frequency conversion module are connected with the CPU; the PCB is connected with the CPU through the detecting sensor socket;
the frequency conversion module controlled by the CPU will produce different frequency ranges according to the gears chosen and implement scanning with increasing frequency within the chosen range.

2. The dental implant detector using constant current source according to claim 1, wherein the PCB base is made with glass fiber cloth material (FR-4), which can be 1.0 mm, 1.2 mm or 1.5 mm thick;
the copper line constituting a coil is 35 um thick and 0.2 mm-0.25 mm wide; the interval between two neighboring coil rings is 0.1 mm-0.15 mm; the detecting coil on the layer of the PCB base has 6 rings, and the inner ring is smaller than the external ones; the coil is connected through a via hole with the coil on the plane of the PCB base; the coil on the plane is also made with 6 rings which become larger from inside out; the center line of the coil on the plane corresponds in position with the center line of intervals between coil rings on the layer;
the detecting coil on the layer measures 5.5 mm-6.6 mm longitudinally and 6.5 mm-7.6 mm laterally; the straight part of a ring of the involute racetrack coil is 2 mm-3 mm long;
the diameter of the semicircular part of the involute racetrack coil is 2.5 mm, 3.1 mm, 3.7 mm, 4.3 mm, 4.9 mm and 5.5 mm, from inside out, with a positive variation of 0-0.1 mm;
the lateral dimension of the involute racetrack coil is 3.5 mm, 4.1 mm, 4.7 mm, 5.3 mm, 5.9 mm and 6.5 mm, from inside out, with a positive variation of 0-0.1 mm;
the center hole on the PCB Base measures 2 mm-3 mm in diameter, which is laminated with copper around;
the three via holes connecting coils on the layer and plane of the PCB base measure 500 um-700 um in diameter, and are distributed along an obtuse triangle.

3. The dental implant detector using constant current source according to claim 1, wherein the detecting coil on the layer of PCB base produces concave magnetic field distribution; the sinusoidal AC flowing through the above coil can be adjusted between 230 kHz and 240 kHz in frequency, and its peak voltage will fluctuate between 400 mV and 450 mV during normal work.

4. The dental implant detector using constant current source according to claim 1, wherein the detecting sensor socket can rotate 0°-90°, 90°-180° and 180°-270° inside the implant locator; the detecting sensor socket is inserted into the plughole of the implant locator;
the plugging end of the detecting sensor socket takes a cylindrical shape, which has a limit boss at 0° position; there are rhombic spring leaves covering the plugging end on the outside; the spring leaves are locked inside the plughole by limit steps set up on the inner wall of the plughole, where, locating slots are also used to harbor 4 corners of the rhombic spring leaves; the angle between two neighboring limit steps is 90°;
the detecting sensor has a hollow structure and there are electrical cables mounted inside.

5. The dental implant detector using constant current source according to claim 1, wherein the status display control module is located where the implant locator bends; the module is split into 8 parts, with each part being equipped with a red LED light and a green one; the 8 parts are display areas 0-5 and key 1 and key 2; the circular display area 0 is positioned at the center, around which surround Key 1 and key 2; there is a circular area comprising display areas 1-5 outside Key 1 and Key 2.

6. The dental implant detector using constant current source according to claim 1, wherein it is powered by battery, which is mounted inside the battery compartment at the end of implant locator and provides electricity to both the detecting sensor and the implant locator;
when the battery voltage nears its critical point, the constant voltage module controlled by the CPU will turn on the constant voltage circuit to provide for normal function of the device and reduce under-performance time. When battery voltage falls below the threshold value, the device cannot start and a reminder of replacing the battery shows up;
the frequency conversion module has 3 gears: the high gear of a 8-12 MHz frequency range, the intermediate gear of 4-8 MHz, and the low gear of 0.1-4 MHz. A 300 um thick copper mesh shield is embedded on the inner wall of the implant locator.

7. A dental implant detecting method using constant current source has the following characteristics:
Step 1. put a battery inside the battery compartment of the implant locator, insert the detecting sensor and the sensor socket into the front end of the implant locator;
Step 1. press Key 2, the buzzer gives a short beep, green LED lights at display areas 0-5 will be lighted for 0.5 second before going off, after which, the implant locator starts self-checking. If the locator fails in self-checking, display areas 1-5 will light the red LED lights successively with an interval of 0.2 second before the locator turns off automatically in 1 second; if the locator passes self-checking, display areas 1-5 will light the green LED lights constantly;
Step 3. press Key 1, the system enters into treatment mode, and display areas 0-5 twinkle their green LED lights simultaneously with an interval of 0.5 second in a cyclic way; when the detecting sensor enters a moderate or weak detecting area, green and red LED lights at display areas 0-5 will be turned on simultaneously to show an orange color; when the detecting sensor enters a strong detecting area, display area 0 will ignite its green LED light and the buzzer will give off two long beeps, which indicates the implant is found; after marking the position, one can continue to find the next implant.

8. The dental implant detecting method using constant current source according to claim 7, during the above-mentioned step 3, the following frequency conversion operations are also implemented:
send the detecting sensor into the oral cavity, rotate the detecting sensor socket to get the best angle for carrying out detection; press down the treatment key, namely Key 1; shift to the low frequency gear and move the sensor over tooth surface; if the sensor enters into the implantation area, display areas 1-5 will be lighted up with an orange color due to the green and red lights being turned on simultaneously; continue to move the sensor, and when the sensor comes to the center of the implantation area, the green LED light goes on at display area 0 while lights at other display areas are off; if no implant is found, shift to intermediate or high gears for higher frequency produced by the frequency conversion module under control of CPU to re-detect until the implant is found.

9. The dental implant detecting method using constant current source according to claim 7, the above-mentioned step 3 also include the following detecting sub-steps:
Step 3.1. insert the coil-mounting PCB base into probe of the detector and send the probe into vicinity of teeth in oral cavity;
Step 3.2. start power supply to the detector, and high-frequency electrical current will flow through the detecting coil and produce around the coil a magnetic field with periodical oscillation;
Step 3.3. when the magnetic field interacts with the metallic dental implant buried under the gum, it will produce induced electromotive force inside the implant, as a result of which, an induced current is generated; however, magnetic field of the induced current will interfere with the original magnetic field around the coil to bring about changes to electrical current in the coil; the testing device from the detector will give signals to warn of changes of the current;
since coils on the layer and plane of the PCB base are asymmetric, their magnetic fields differ in intensity; even at the middle between teeth of the upper and lower jaws, the probe can still detect the metallic implant quickly, and then locate it accurately by moving the probe.

10. The dental implant detecting method using constant current source according to claim 9, magnetic induction intensity of the involute racetrack coils in step 3 is calculated with the formula:
$$\begin{matrix}\vec{B} ( z ) = \frac{{μ}_{0} I}{4 π} {∳}_{l} \frac{{dl}^{′} \times \vec{R}}{{R}^{3}} \\ = \frac{{μ}_{0} Ia}{4 π {R}^{2}} [ {\int }_{0}^{2 π} {\vec{e}}_{φ} \times ( - {e}_{r} sin α + {e}_{z} cos α ) d {φ}^{′} ] \\ = \frac{{μ}_{0} Ia}{4 π {R}^{2}} ( {\vec{e}}_{z} sin α {\int }_{0}^{2 π} d {φ}^{′} + cos α {\int }_{0}^{2 π} {\vec{e}}_{r} d {φ}^{′} ) \\ = {\vec{e}}_{z} \frac{{μ}_{0} {Ia}^{2}}{2 {R}^{3}} \\ = {\vec{e}}_{z} \frac{{μ}_{0} {Ia}^{2}}{{2 [ {a}^{2} + {z}^{2} ]}^{3 / 2}}\end{matrix}$$
taking coil on the layer as an xoy plane, origin of coordinates is at the middle of the center hole, therefore, magnetic field distribution along the central axis above the xoy plane is calculated with the formula:
$${\vec{B} ( z )}_{up} = {\vec{e}}_{z} \frac{{μ}_{0} {Ia}^{2}}{2} [ \frac{1}{{[ {a}^{2} + {z}^{2} ]}^{\frac{3}{2}}} + \frac{1}{{[ {( a + Δ a )}^{2} + {z}^{2} ]}^{\frac{3}{2}}} + \frac{1}{{[ {( a + 2 Δ a )}^{2} + {z}^{2} ]}^{\frac{3}{2}}} + \frac{1}{{[ {( a + 3 Δ a )}^{2} + {z}^{2} ]}^{\frac{3}{2}}} + \frac{1}{{[ {( a + 4 Δ a )}^{2} + {z}^{2} ]}^{\frac{3}{2}}} + \frac{1}{{[ {( a + 5 Δ a )}^{2} + {z}^{2} ]}^{\frac{3}{2}}} ]$$
magnetic field distribution along the central axis below the xoy plane is calculated with the formula:
$${\vec{B} ( z )}_{down} = {\vec{e}}_{z} \frac{{μ}_{0} {Ia}^{2}}{2} [ \frac{1}{{[ {( a + Δ a )}^{2} + {( z + d )}^{2} ]}^{\frac{3}{2}}} + \frac{1}{{[ {( a + 2 Δ a )}^{2} + {( z + d )}^{2} ]}^{\frac{3}{2}}} + \frac{1}{{[ {( a + 3 Δ a )}^{2} + {( z + d )}^{2} ]}^{\frac{3}{2}}} + \frac{1}{{[ {( a + 4 Δ a )}^{2} + {( z + d )}^{2} ]}^{\frac{3}{2}}} + \frac{1}{{[ {( a + 5 Δ a )}^{2} + {( z + d )}^{2} ]}^{\frac{3}{2}}} + \frac{1}{{[ {( a + 6 Δ a )}^{2} + {( z + d )}^{2} ]}^{\frac{3}{2}}} ]$$
finally, overall magnetic field distribution along the central axis is calculated with the formula:
$${\vec{B} ( z )}_{total} = {\vec{e}}_{z} \frac{{μ}_{0} {Ia}^{2}}{2} [ \frac{1}{{[ {a}^{2} + {z}^{2} ]}^{\frac{3}{2}}} + \frac{1}{{[ {( a + Δ a )}^{2} + {z}^{2} ]}^{\frac{3}{2}}} + \frac{1}{{[ {( a + 2 Δ a )}^{2} + {z}^{2} ]}^{\frac{3}{2}}} + \frac{1}{{[ {( a + 3 Δ a )}^{2} + {z}^{2} ]}^{\frac{3}{2}}} + \frac{1}{{[ {( a + 4 Δ a )}^{2} + {z}^{2} ]}^{\frac{3}{2}}} + \frac{1}{{[ {( a + 5 Δ a )}^{2} + {z}^{2} ]}^{\frac{3}{2}}} ] + {\vec{e}}_{z} \frac{{μ}_{0} {Ia}^{2}}{2} [ \frac{1}{{[ {( a + Δ a )}^{2} + {( z + d )}^{2} ]}^{\frac{3}{2}}} + \frac{1}{{[ {( a + 2 Δ a )}^{2} + {( z + d )}^{2} ]}^{\frac{3}{2}}} + \frac{1}{{[ {( a + 3 Δ a )}^{2} + {( z + d )}^{2} ]}^{\frac{3}{2}}} + \frac{1}{{[ {( a + 4 Δ a )}^{2} + {( z + d )}^{2} ]}^{\frac{3}{2}}} + \frac{1}{{[ {( a + 5 Δ a )}^{2} + {( z + d )}^{2} ]}^{\frac{3}{2}}} + \frac{1}{{[ {( a + 6 Δ a )}^{2} + {( z + d )}^{2} ]}^{\frac{3}{2}}} ]$$
